(19) United States
(12) Patent Application Publication
(10) Pub. No.: US 2019/0331653 A1
(43) Pub. Date: Oct. 31, 2019

(54) SYSTEM AND METHOD FOR EGG QUALITY CONTROL

(71) Applicant: Walmart Apollo, LLC, Bentonville, AR (US)

(72) Inventors: Benjamin D. ENSSLE, Bella Vista, AR (US); Shaun R. RORRISON, Bentonville, AR (US); Ross A. MCKINNON, Rogers, AR (US); David B. BRIGHTWELL, Bentonville, AR (US)

(73) Assignee: Walmart Apollo, LLC, Bentonville, AR (US)

(21) Appl. No.: 16/397,262

(22) Filed: Apr. 29, 2019

Related U.S. Application Data
(60) Provisional application No. 62/663,592, filed on Apr. 27, 2018.

(51) Int. Cl. G01N 33/08; A01K 43/08; A01K 43/06; G01N 23/04; G01N 21/25; G01N 9/36

(52) U.S. Cl. CPC G01N 33/08; A01K 43/08; G01N 9/36; G01N 23/04; G01N 21/253; A01K 43/06

(57) ABSTRACT

Systems and methods for controlling egg quality are provided. An example method can include: moving a plurality of cartons packed with eggs by a conveyor belt; measuring a weight of each carton to decide whether the weight of each carton is within a carton weight tolerance; detecting a moisture level inside the carton to decide whether the moisture level of the carton is within a moisture level tolerance; determining whether a shell of the egg is intact and a volume of egg yolk and white is within a volume tolerance; detecting a color of each egg in the carton to decide whether the colors of the eggs are consistent; measuring a weight of the egg in the carton to determine whether the weight of the egg is within an egg weight tolerance; and measuring a density of the egg to determine whether the egg is within a density tolerance.

### CROSS REFERENCE TO RELATED APPLICATION

This patent application claims the benefit of U.S. Provisional Application No. 62/663,592, filed on Apr. 27, 2018, content of which is incorporated by reference herein.

### BACKGROUND

### 1. Technical Field

The present disclosure relates to systems and methods for egg quality control.

### 2. Introduction

Wholesalers and retailers receive eggs packed in cartons from an egg supplier. However, some eggs in the cartons may be broken during transportation. Some eggs may look good but not meet a predefined quality requirement before being placed in the merchant's stores for sale. Egg quality needs to be maintained and evaluated for providing good quality eggs in each carton for consumers to purchase. Generally, if at least one egg in a carton is identified to be unacceptable by a merchant, the entire carton of eggs is usually discarded, which may lead to a big waste of money for the merchant. There is a need to have an advanced system to conduct quality control for each egg and to select good quality or qualified eggs for customers. Moreover, accurate data about acceptable and unacceptable eggs received from the egg supplier should be obtained in order to manage an inventory and to charge back the egg supplier for the unqualified eggs.

### SUMMARY

An example system configured for controlling egg quality according to the concepts and principles disclosed herein can include: a conveyor belt configured to move a plurality of cartons, each of the plurality of cartons being filled with a number of eggs; a weight sensor disposed under the conveyor belt and configured to measure a weight of each carton to decide whether the weight of each carton is within a carton weight tolerance; a moisture sensor disposed under the conveyor belt and configured to detect a moisture level inside the carton to decide whether the moisture level of the carton is within a moisture level tolerance; an x-ray sensor disposed under the conveyor belt and configured to perform an image analysis of each egg in the carton to determine whether a shell of the egg is intact and a volume of egg yolk and white is within a volume tolerance; a color sensor disposed under the conveyor belt and configured to detect a color of each egg in the carton to decide whether colors of the eggs in the carton are consistent; a scale disposed above the conveyor belt and configured to measure a weight of the egg in the carton to determine whether the weight of the egg is within an egg weight tolerance, the egg being lifted by an air vacuum suction cup; and a density sensor disposed under the conveyor belt and configured to measure a density of the egg to determine whether the egg is within a density tolerance.

An example method of performing concepts disclosed herein can include receiving a plurality of cartons at a conveyor belt and moving the cartons along the conveyor belt to a plurality of sensing stations, each of the plurality of cartons being filled with a number of eggs; measuring, via a weight sensor at a carton weight sensing station, a weight of each carton to decide whether the weight of each carton is within a carton weight tolerance; detecting, via a moisture sensor at a carton moisture sensing station, a moisture level inside the carton to decide whether the moisture level of the carton is within a moisture level tolerance; performing, via an x-ray sensor at an egg x-ray sensing station, an image analysis of each egg in the carton to determine whether a shell of the egg is intact and a volume of egg yolk and white is within a volume tolerance; detecting, via a color sensor at an egg color sensing station, a color of each egg in the carton to decide whether colors of the eggs in the carton are consistent; measuring, via a scale at an egg weight sensing station, a weight of the egg in the carton to determine whether the weight of the egg is within an egg weight tolerance; and measuring, via a density sensor at an egg density sensing station, a density of the egg to determine whether the egg is within a density tolerance.

An example non-transitory computer-readable storage medium configured as disclosed herein can have instructions stored which, when executed by a processor, cause the processor to perform operations including moving a plurality of cartons by a conveyor belt, each of the plurality of cartons being filled with a number of eggs; measuring, via a weight sensor, a weight of each carton to decide whether the weight of each carton is within a carton weight tolerance; detecting, via a moisture sensor, a moisture level inside the carton to decide whether the moisture level of the carton is within a moisture level tolerance; performing, via an x-ray sensor, an image analysis of each egg in the carton to determine whether a shell of the egg is intact and a volume of egg yolk and white is within a volume tolerance; detecting, via a color sensor, a color of each egg in the carton to decide whether colors of the eggs in the carton are consistent; measuring, via a scale, a weight of the egg in the carton to determine whether the weight of the egg is within an egg weight tolerance, the egg being lifted by an air vacuum suction cup; and measuring, via a density sensor, a density of the egg to determine whether the egg is within a density tolerance.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims, or can be learned by the practice of the principles set forth herein.

### BRIEF DESCRIPTION OF THE DRAWINGS

Example embodiments of this disclosure are illustrated by way of an example and not limited in the figures of the accompanying drawings, in which like references indicate similar elements and in which:

FIG. 1 is a diagram illustrating an example system of conducting egg quality control which some example embodiments may be implemented;

FIG. 2 shows a diagram illustrating a module for lifting an egg in accordance with some example embodiments;

FIG. 3 is a flowchart diagram illustrating a process for examining egg quality in accordance with some example embodiments; and

FIG. 4 is a block diagram illustrating an example computer system in which some example embodiments may be implemented.

It is to be understood that both the foregoing general description and the following detailed description are example and explanatory and are intended to provide further explanations of the invention as claimed only and are, therefore, not intended to necessarily limit the scope of the disclosure.

### DETAILED DESCRIPTION

Various example embodiments of the present disclosure will be described in detail below with reference to the accompanying drawings. Throughout the specification, like reference numerals denote like elements having the same or similar functions. While specific implementations and example embodiments are described, it should be understood that this is done for illustration purposes only. Other components and embodiments may be used without parting from the spirit and scope of the disclosure.

The present disclosure provides systems and methods for performing quality control for a large quantity of eggs. The systems and methods described in the present disclosure may also allow for identifying acceptable eggs and unacceptable eggs received from an egg supplier. Payment for the total of eggs received may be adjusted based on the number of acceptable eggs. Moreover, an inventory may be more accurately managed and forecasted. The systems and methods may also be used to sort eggs into cartons of a particular type, e.g., jumbo brown eggs.

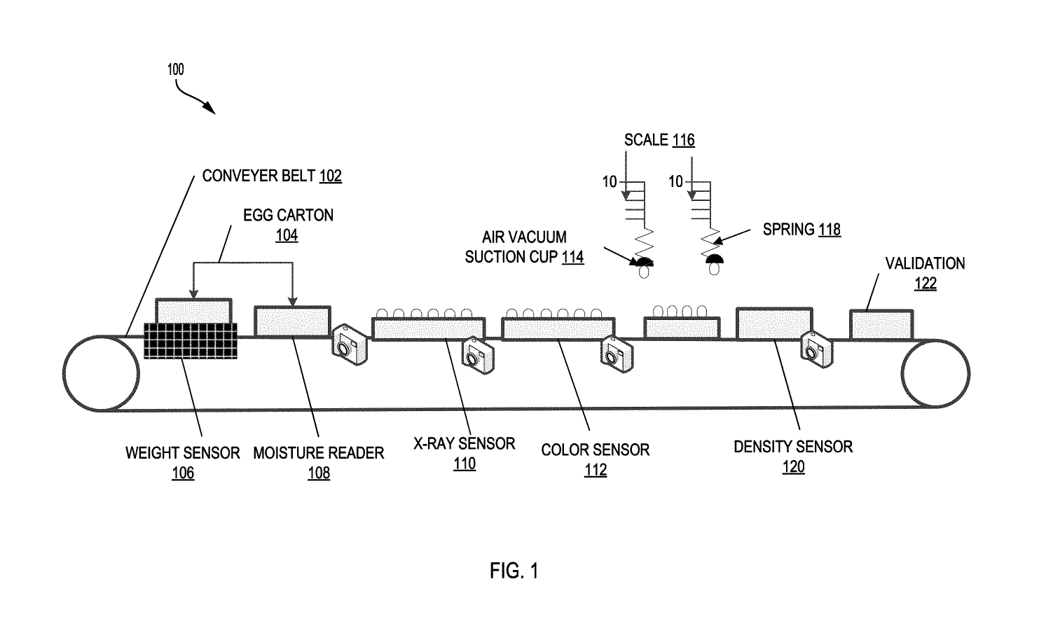

FIG. 1 is a diagram illustrating an example system 100 for egg quality control in which some example embodiments may be implemented. The example system 100 may include a plurality of sensors and measuring components for detecting quality of eggs. The example system 100 may include one or more of a conveyer system, weight sensor 106, moisture reader 108, x-ray sensor 110, color sensor 112, air vacuum suction cups 114, scale 116, spring 118, density sensor 120, validation module 122, and one or more processors (not shown).

The conveyer system may include one or more conveyor belts 102 for moving a plurality of eggs through different detecting stations where different parameters of the eggs may be detected and measured. In some example embodiments, the plurality of the sensors may include digital cameras to perform different egg quality measurements and evaluations. The one or more processors may be coupled to the sensors and be configured to control the cameras or sensors to conduct different operations for detecting the quality of eggs. The plurality of sensors may include memories to store one or more sets of instructions executable by the one or more processors as well as other sensor data used by the processors. The one or more processors may be integrated in or connected to various mechanical components to facilitate multiple functionalities of the egg quality control through the different detecting stations.

Eggs may be graded and classified by color, (white or brown), and size, (jumbo, extra-large, large, medium, small, etc.). An acceptable egg may have a size, a color and a weight within a set of tolerances. The set of the tolerances for each egg or each carton of eggs may be predefined based on an egg size and a total number of eggs in each carton. Different tolerances may be predefined and associated with different sizes of eggs. The tolerances for a certain type of carton of eggs may include various parameters, such as a carton weight, a moisture level, an egg shell structure, a volume of egg yolk and egg white, an egg color, an egg weight, an egg density, etc.

In some example embodiments, the eggs may be packed in a plurality of egg cartons 104. The cartons 104 are received and loaded onto the conveyor belt 102. The cartons 104 may be opened or closed. The conveyor belt 102 may be controlled by a processor to load and move a plurality of cartons 104 for checking egg quality. In some cases, the plurality of cartons 140 may be marked by a supplier with a code printed on the carton. The code may include information of a number of eggs and an egg size. The system 100 may first read the bar code to obtain the egg size and the total number of eggs in the carton 104 for further egg quality examination. A corresponding set of requirements for each carton may be determined for the egg quality examination. The plurality of cartons of eggs are examined to ensure the eggs are of sufficient quality to be sold to consumers. Eggs that do not meet requirements are removed from the carton.

In some example embodiments, the egg cartons 104 may be placed on a pallet to keep the cartons stable while the egg cartons 104 are moved along the conveyor belt 102. The pallet may be placed on a foam or air cushion which moves with the conveyor belt 102 through the different stations.

The weight sensor 106 may be positioned under the conveyor belt 102 to detect and measure a weight of a pallet or carton of eggs while the cartons of eggs move along the conveyor belt 102. The weight sensor 106 may detect a weight loss (e.g., weight imbalance) of the carton 104 due to leaking eggs or missing eggs inside the carton 104 by measuring the carton weight and comparing the carton weight to a predefined carton weight tolerance. Cartons 104 that do not meet a predefined carton weight tolerance are removed from the conveyor belt 102.

The moisture reader 108 may include a moisture sensor to detect and measure a moisture level in a carton of eggs moving along the conveyor belt 102. The moisture reader 108 may determine whether there is a broken or leaking egg in the carton 104. The moisture reader 108 may be an imaging moisture meter including a digital camera, a moisture sensor, a processor, and a memory for detecting and measuring a moisture level in a carton. For example, when the moisture level in the air humidity of the closed carton exceeds a moisture tolerance, e.g., 20%, it may indicate the carton is wet and there is one or more broken or leaking eggs in the carton.

The X-ray sensor 110 may examine the egg shell and a volume of the egg yolk and white. The X-ray sensor 110 may be integrated in or coupled with a digital camera to perform an image analysis to detect a structure and thickness of the egg shell and a position of yolk and white in the egg. The X-ray sensor may be used for volume, size and shape validation in order to ensure eggs meet standards of egg volume, size, and shape. For example, the egg yolk of a good quality egg should be well centered. A large egg should be within a particular volume tolerance based on predefined rules or specifications. In some example embodiments, the information obtained by the x-ray sensor 110 may be used to determine whether the egg has a proper shape and size. Customers may not like irregular shaped eggs or irregular sizes of eggs in a carton. If an egg has an irregular shape and size or non-uniform shell thickness, the system 100 may remove the egg from the carton 104.

The color sensor 112 may detect if each of the eggs in the carton 104 has a proper color. The color sensor 112 may be coupled with a digital camera to perform an image analysis and recognition related to volume, size and shape validation to ensure that the eggs meet the predefined color standard and are shaped as expected. The system 100 may place the eggs with one color and one size together into a new carton. For example, large white eggs should only be placed in cartons designated for large white eggs and jumbo brown eggs should only be placed in cartons designated for jumbo brown eggs. The color sensor 112 may also be used to check whether the egg has stains. The eggs with stains may be removed from the carton. In one example embodiment, the camera used for egg volume, size, and shape validation may be the same camera used with the color sensor 112.

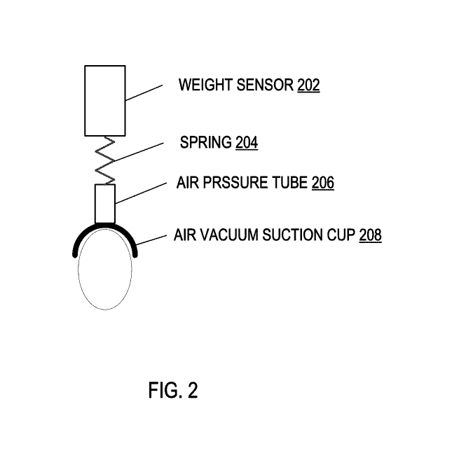

The scale 116 may measure the weight of an egg. Air vacuum suction cup 114 may automatically lift the egg from the carton for the scale 116 to weigh the egg. The scale 116 may be a spring scale. FIG. 2 illustrates an example of lifting an egg by an air vacuum suction cup 208 and measuring the egg weight in accordance with some example embodiments. A first end of a spring 204 may be connected to the weight sensor 202 and a second end of the spring 204 may be connected to an air pressure tube 206. The weight sensor 202 may be coupled to or be integrated in the scale 116 for measuring the egg weight. The air pressure tube 206 may be connected to the air vacuum suction cup 208. The air vacuum suction cup 208 may be configured to drop the egg back to the carton 104 if the egg weight is within the weight tolerance.

A density sensor 120 may perform an image analysis for measuring a content density of the egg in the carton 104. An egg content density is a ratio of an egg mass to an egg volume. The density sensor 120 may be coupled to or integrated in a digital camera with a processor and a memory to perform an image analysis related to volume and mass calculation and to ensure that the eggs meet the predefined size standard and are shaped as expected. Each egg with a certain size may need to meet a predefined content density standard.

The validation module 122 may be used to validate the eggs for acceptance as qualified eggs. Qualified eggs in cartons may be moved, sorted and packed to make full cartons of eggs with same size and color, for example, each holding 18 eggs, 12 eggs, or 6 eggs. The cartons fully packed with the qualified eggs may be labeled with grade, size, color, weight, producer, expiration date, etc. Labels may be printed directly on cartons or attached to the cartons.

In some example embodiment, the system 100 may include a designated subsystem to process eggs removed from the conveyor belt 102. These eggs may not be the broken eggs, but have certain parameters which are not within at least one of the predefined tolerances. For example, some eggs may be too big or too small and may not within a weight tolerance. The colors of some eggs may not be within a color tolerance. Those eggs are not qualified to be sold to the customers. Since those eggs are not broken, they may be collected for other purposes.

In some example embodiments, the example system 100 may include a calculation module to calculate the numbers of qualified eggs and unqualified eggs received from the egg supplier based on the results of the egg quality examination through the different detecting stations. For example, the merchant may receive a total of 10,000 eggs from an egg supplier, and 1,500 eggs. e.g., 15% of the eggs, are determined to be unqualified during the egg quality control process. The merchant system may be able to adjust payment for the total of eggs received from the egg supplier and charge back to the egg supplier for the unqualified eggs. Moreover, the merchant may be able to accurately manage the inventory and determine a number of additional eggs to be ordered for fulfilling potential needs in the inventory, taking into account the percentage of unqualified eggs based on historical performance.

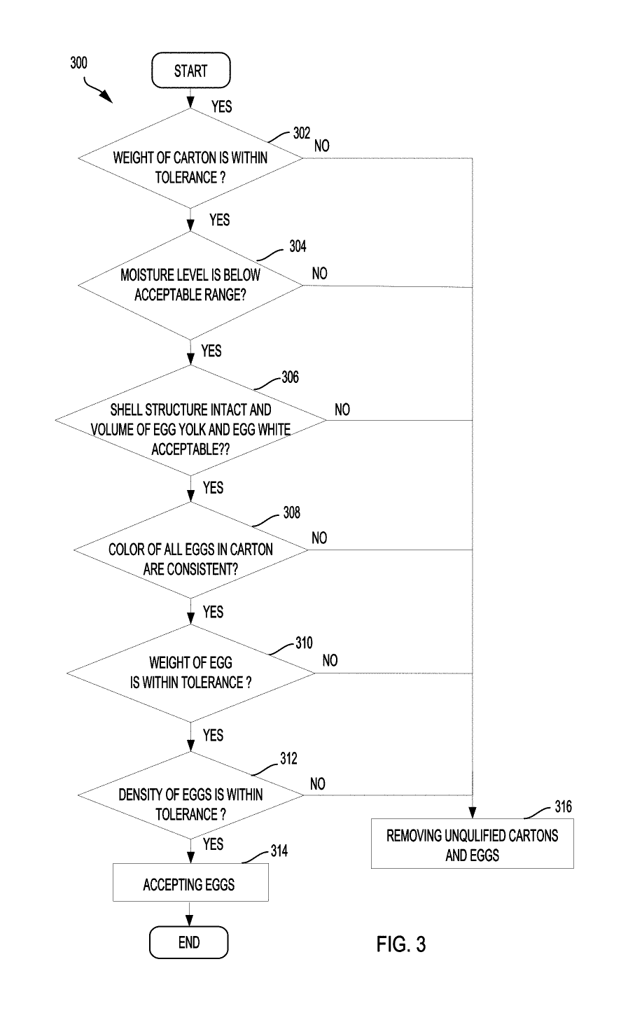

FIG. 3 is a flowchart diagram illustrating an example process 300 for determining egg quality in accordance with some example embodiments. The process 300 may be implemented in the above described systems and may include one or more of the following steps in any combination.

In step 302, the weight sensor 106 may detect and measure a weight of each of the plurality of cartons and decide whether the weight of a carton is within a carton weight tolerance. The plurality of cartons may be closed during the operation. If the weight of the carton is measured to be less than a carton weight tolerance, it may indicate there is one or more broken, missing, or incorrect size eggs inside the carton. The information obtained by the weight sensor 106 may be sent to the one or more processors for controlling the corresponding mechanical components to remove the whole carton from the conveyor belt 102. The cartons of eggs may be removed as disposal of product and charged back to supplier. In one example embodiment, the eggs in the removed cartons may be individually considered for quality control. The carton within the carton weight of the tolerance may remain on the conveyor belt 102 for further processing.

In step 304, the moisture reader 108 may detect and measure a moisture level inside a carton and decide whether there is a broken or leaking egg in the carton. The carton may be a closed egg carton or be opened during the operation. If the detected moisture level in the carton is below a moisture level tolerance, there may be one or more broken or leaking eggs in the carton. The information obtained by moisture reader 108 may be sent to the one or more processors for controlling the corresponding mechanical components to remove the whole carton or the broken or leaking eggs from the conveyor belt 102. The removed cartons of eggs may be considered as disposal of product and charged back to supplier. The carton having the moisture level within an acceptable range of the tolerance may remain on the conveyor belt 102 for further processing.

In step 306, the x-ray sensor 110 may perform an image analysis and recognition for each egg in a carton. The plurality of cartons may be opened for the x-ray sensor to detect and determine whether a shell of each egg is intact and whether a volume of the egg yolk and egg white is within a predefined volume tolerance. If the egg shell of an egg in the carton is not intact or the volume of egg yolk and egg white is not within the volume tolerance, the information obtained by the x-ray sensor 110 may be sent to the one or more processors for controlling the corresponding mechanical components to remove the egg from the carton. The unqualified egg detected by the x-ray sensor 110 may be removed and charged back to supplier. The unqualified eggs may be removed by air vacuum suction cups 114. The eggs detected to be intact and to have the volume of egg yolk and egg white within a range of the volume tolerance may remain on the conveyor belt 102 for further processing.

In step 308, the color sensor 112 may detect whether each of the eggs in a carton has a proper brown color or a proper white color within the predefined color tolerance. If the color of an egg in the carton is not within the color tolerance, the information obtained by the color sensor 112 may be sent to the one or more processors for controlling the corresponding mechanical components to remove the egg from the carton. The unqualified eggs may be removed by the air vacuum suction cups 114. The eggs with the same color may be arranged to ensure the colors of all eggs in a carton are consistent. White eggs may only be in a white egg carton and brown eggs may only be in a brown egg carton. For example, brown eggs removed from white egg cartons may be grouped together to make a new carton of brown eggs.

In step 310, the scale 116 may measure and determine whether a weight of one or more of the eggs in a carton is within a weight tolerance. The individual egg weight may be measured with the spring scale 116 using air-powered suction cups lifting eggs. The eggs having a weight within an acceptable tolerance may be dropped back by the air vacuum suction cups 114 in the carton on the conveyor belt 102 for further processing. The unqualified eggs that do not meet the weight tolerance may be removed as disposal of product and charged back to supplier. In one example embodiment, the unqualified eggs that do not meet the weight tolerance may be removed by the air vacuum suction cups 114 to a designated subsystem for further processing.

In some example embodiments, an egg in the carton may be randomly selected as a sample for all eggs in the whole carton and be measured to determine whether it is within a weight tolerance. If the weight of the randomly selected egg is less than a predefined weight of the tolerance, the whole carton may be removed and charged back to supplier. In one example embodiment, the whole carton may be removed to a designated subsystem for further processing.

In step 312, the density sensor 120 may be controlled to measure the density of each egg in a carton to determine whether the density of each egg is within a predefined density. If the density of each egg in the carton is not within the density tolerance, the egg may be unqualified and be removed, and charged back to supplier. The eggs having the density within the range of the density tolerance may remain on the conveyor belt 102 for further processing. If the density of an egg in the carton is not within the density tolerance, the information obtained by the density sensor 120 may be sent to the one or more processors for controlling the corresponding mechanical components to remove the egg from the carton. The unqualified eggs may be removed by air vacuum suction cups 114.

In step 314, the eggs having various parameters within the predefined tolerances may be accepted. The accepted and qualified eggs in each of the plurality of detected cartons may be validated via a validation module and regrouped into cartoons such that each cartoon is fully filled with eggs with same size and color.

In step 316, the number of the unqualified eggs being removed from the conveyer belt 102 may be counted and disposed. The corresponding prices of the unqualified eggs may be calculated for charging back to the egg supplier. The numbers of qualified eggs may be calculated based on the results of the egg quality examination through the different detecting stations. The merchant may analyze their store egg sale records along with the numbers of qualified eggs in order to accurately manage the inventory and determine a number of additional eggs to be ordered for fulfilling potential needs in the inventory.

In some example embodiments, the merchant may receive bulks of eggs packed in different sizes of filler trays and other packages from an egg supplier. The example system 100 may continuously conduct quality control for thousands of eggs during a period of time and then sort and pack good quality eggs with different sizes of cartons for selling them at the merchant's store. First, the example system 100 may receive egg information, e.g., producer and expiration data, before evaluating the eggs. Secondly, the example system 100 may use a plurality of digital cameras coupled with a plurality of sensors to detect and measure parameters of each egg including moisture, weight, color, size, shape, density, etc. Data processing may be performed by one or more processors to identify the good quality eggs by comparing the detected egg parameters to the set of predefined tolerances. The example system 100 may further include a robotic system including mechanical equipment and components to remove the unqualified eggs out of the processing system and grade and pack the good quality eggs in different sizes of cartons based on size, color, weight, etc. For example, the good quality eggs may first be graded as color, then the good quality eggs with the same color may be graded as different sizes. Finally, a certain color, size, weight of eggs may be packaged into different sizes of cartons and being labeled properly. The example system 100 may timely calculate the numbers of qualified eggs and unqualified eggs received from the egg supplier during the egg quality detection process. The data of the qualified eggs and unqualified eggs obtained during the egg quality detection process may be used for accurate inventory management, fee calculation and adjustment of the bulks of eggs from the egg supplier.

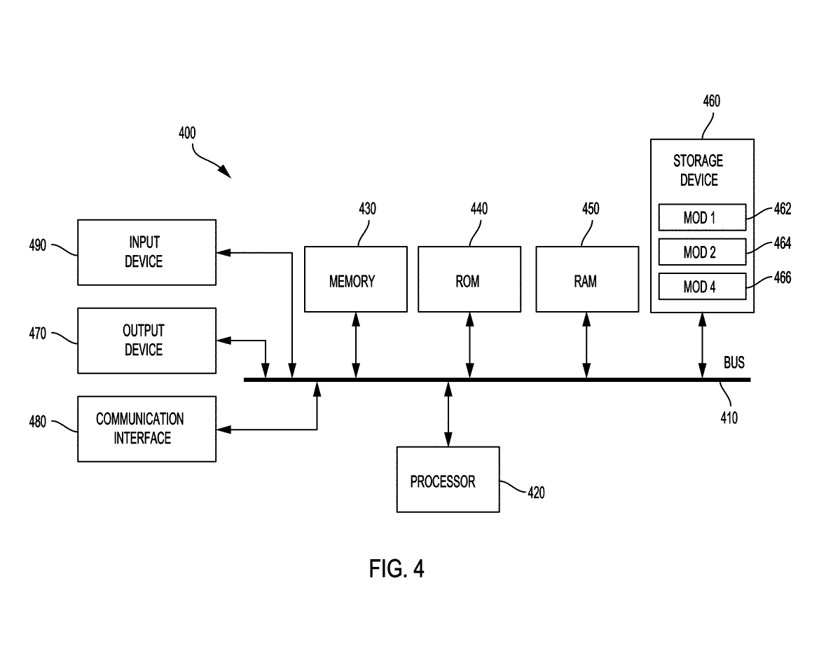

FIG. 4 illustrates an example computer system 400 which can be used to perform the processes for egg quality control as disclosed herein. The exemplary system 400 can include a processing unit (CPU or processor) 420 and a system bus 410 that couples various system components including the system memory 430 such as read only memory (ROM) 440 and random access memory (RAM) 450 to the processor 420. The system 400 can include a cache of high speed memory connected directly with, in close proximity to, or integrated as part of the processor 420. The system 400 copies data from the memory 430 and/or the storage device 460 to the cache for quick access by the processor 420. In this way, the cache provides a performance boost that avoids processor 420 delays while waiting for data. These and other modules can control or be configured to control the processor 420 to perform various actions. Other system memory 430 may be available for use as well. The memory 430 can include multiple different types of memory with different performance characteristics. It can be appreciated that the disclosure may operate on a computing device 400 with more than one processor 420 or on a group or cluster of computing devices networked together to provide greater processing capability. The processor 420 can include any general purpose processor and a hardware module or software module, such as module 1 462, module 2 464, and module 3 466 stored in storage device 460, configured to control the processor 420 as well as a special-purpose processor where software instructions are incorporated into the actual processor design. The processor 420 may essentially be a completely self-contained computing system, containing multiple cores or processors, a bus, memory controller, cache, etc. A multi-core processor may be symmetric or asymmetric.

The system bus 410 may be any of several types of bus structures including a memory bus or memory controller, a peripheral bus, and a local bus using any of a variety of bus architectures. A basic input/output (BIOS) stored in ROM 440 or the like, may provide the basic routine that helps to transfer information between elements within the computing device 400, such as during start-up. The computing device 400 further includes storage devices 460 such as a hard disk drive, a magnetic disk drive, an optical disk drive, tape drive or the like. The storage device 460 can include software modules 462, 464, 466 for controlling the processor 420. Other hardware or software modules are contemplated. The storage device 460 is connected to the system bus 410 by a drive interface. The drives and the associated computer-readable storage media provide nonvolatile storage of computer-readable instructions, data structures, program modules and other data for the computing device 400. In one aspect, a hardware module that performs a particular function includes the software component stored in a tangible computer-readable storage medium in connection with the necessary hardware components, such as the processor 420, bus 410, display 470, and so forth, to carry out the function. In another aspect, the system can use a processor and computer-readable storage medium to store instructions which, when executed by the processor, cause the processor to perform a method or other specific actions. The basic components and appropriate variations are contemplated depending on the type of device, such as whether the device 400 is a small, handheld computing device, a desktop computer, or a computer server.

Although the exemplary embodiment described herein employs the hard disk 460, other types of computer-readable media which can store data that are accessible by a computer, such as magnetic cassettes, flash memory cards, digital versatile disks, cartridges, random access memories (RAMs) 450, and read only memory (ROM) 440, may also be used in the exemplary operating environment. Tangible computer-readable storage media, computer-readable storage devices, or computer-readable memory devices, expressly exclude media such as transitory waves, energy, carrier signals, electromagnetic waves, and signals per se.

To enable user interaction with the computing device 400, an input device 490 represents any number of input mechanisms, such as a microphone for speech, a touch-sensitive screen for gesture or graphical input, keyboard, mouse, motion input, speech and so forth. An output device 470 can also be one or more of a number of output mechanisms known to those of skill in the art. In some instances, multimodal systems enable a user to provide multiple types of input to communicate with the computing device 400. The communications interface 480 generally governs and manages the user input and system output. There is no restriction on operating on any particular hardware arrangement and therefore the basic features here may easily be substituted for improved hardware or firmware arrangements as they are developed.

The various embodiments described above are provided by way of illustration only and should not be construed to limit the scope of the disclosure. Various modifications and changes may be made to the principles described herein without following the example embodiments and applications illustrated and described herein, and without departing from the spirit and scope of the disclosure.

We claim:

1. A system comprising:
a conveyor belt configured to move a plurality of cartons, each of the plurality of cartons being filled with a number of eggs;
a weight sensor disposed under the conveyor belt and configured to measure a weight of each carton to decide whether the weight of each carton is within a carton weight tolerance;
a moisture sensor disposed along the conveyor belt and configured to detect a moisture level inside the carton to decide whether the moisture level of the carton is within a moisture level tolerance;
an x-ray sensor disposed along the conveyor belt and configured to perform an image analysis of each egg in the carton to determine whether a shell of the egg is intact and a volume of egg yolk and white is within a volume tolerance;
a color sensor disposed along the conveyor belt and configured to detect a color of each egg in the carton to decide whether colors of the eggs in the carton are consistent;
a scale disposed above the conveyor belt and configured to measure a weight of the egg in the carton to determine whether the weight of the egg is within an egg weight tolerance; and
a density sensor disposed along the conveyor belt and configured to measure a density of the egg to determine whether the egg is within a density tolerance.

2. The system of claim 1, wherein performing the image analysis of the egg further comprises detecting a size and a shape of the eggs in the carton and determining whether each egg meets a size and shape tolerance.

3. The system of claim 1, further comprising an air vacuum suction cup is configured to automatically remove a carton from the conveyor belt when the weight of the carton is not within the weight tolerance.

4. The system of claim 1, further comprising an air vacuum suction cup is configured to automatically remove a carton from the conveyor belt when the moisture of the carton is less than a moisture tolerance.

5. The system of claim 1, further comprising an air vacuum suction cup is configured to automatically remove an egg from the carton when the shell of the egg in the carton is not intact or a volume of the egg yolk and white is not within the volume tolerance.

6. The system of claim 1, further comprising an air vacuum suction cup is configured to s automatically remove an egg from the carton when the egg in the carton does meet a size and shape tolerance.

7. The system of claim 1, further comprising an air vacuum suction cup is configured to automatically remove an egg from the carton when the weight of the egg in the carton is detected to be not within a weight tolerance.

8. The system of claim 1, further comprising an air vacuum suction cup is configured to automatically remove an egg from the carton when the density of the egg in the carton is determined to be not within the density tolerance.

9. The system of claim 1, wherein each carton is filled with eggs within the weight, size, color, volume, and density tolerances.

10. The system of claim 1, wherein the system further comprises a calculating module configured to calculate a percentage of the qualified eggs in a total number of eggs in the plurality of cartons and to determine a number of eggs to be ordered for filling up eggs in an inventory.

11. A method, comprising:
receiving a plurality of cartons at a conveyor belt and moving the cartons along the conveyor belt to a plurality of sensing stations, each of the plurality of cartons being filled with a number of eggs;
measuring, via a weight sensor at a carton weight sensing station, a weight of each carton to decide whether the weight of each carton is within a carton weight tolerance;
detecting, via a moisture sensor at a carton moisture sensing station, a moisture level inside the carton to decide whether the moisture level of the carton is within a moisture level tolerance;
performing, via an x-ray sensor at an egg x-ray sensing station, an image analysis of each egg in the carton to determine whether a shell of the egg is intact and a volume of egg yolk and white is within a volume tolerance;
detecting, via a color sensor at an egg color sensing station, a color of each egg in the carton to decide whether colors of the eggs in the carton are consistent;
measuring, via a scale at an egg weight sensing station, a weight of the egg in the carton to determine whether the weight of the egg is within an egg weight tolerance; and
measuring, via a density sensor at an egg density sensing station, a density of the egg to determine whether the egg is within a density tolerance.

12. The method of claim 11, wherein performing the image analysis of the egg further comprises detecting a size and a shape of the egg in the carton and determining whether the egg meets a size and shape tolerance.

13. The method of claim 11, further comprising automatically removing the carton from the conveyor belt when the weight of the carton is not within a carton weight tolerance.

14. The method of claim 11, further comprising automatically removing the carton from the conveyor belt when the moisture level of the carton is not within the moisture level tolerance.

15. The method of claim 11, further comprising automatically removing the egg from the carton when the shell of the egg in the carton is not intact or a volume of the egg yolk is not within the volume tolerance.

16. The method of claim 11, further comprising automatically removing the egg from the carton by an air vacuum suction cup when the weight of the egg in the carton is not within the weight tolerance.

17. The method of claim 11, further comprising automatically removing the egg from the carton when the density of the egg in the carton is not within the density tolerance.

18. The method of claim 11, further comprising:
calculating, by a calculating module, a percentage of qualified eggs in a total number of eggs in the plurality of cartons;
determining a number of eggs to be ordered for filling up eggs in an inventory, wherein each of the qualified eggs is within the weight, size, color, volume, and density tolerances;
determining if any of the initially removed eggs meets the weight, size, color, volume and density requirements for other cartons from which an egg was removed; and
placing the initially removed egg in the another carton.

19. A non-transitory computer-readable storage medium having executed instructions stored which, when executed by a processor, cause the processor to perform operations comprising:
moving a plurality of cartons by a conveyor belt, each of the plurality of cartons being filled with a number of eggs;
measuring, via a weight sensor, a weight of each carton to decide whether the weight of each carton is within a carton weight tolerance;
detecting, via a moisture sensor, a moisture level inside the carton to decide whether the moisture level of the carton is within a moisture level tolerance;
performing, via an x-ray sensor, an image analysis of each egg in the carton to determine whether a shell of the egg is intact and a volume of egg yolk and white is within a volume tolerance;
detecting, via a color sensor, a color of each egg in the carton to decide whether colors of the eggs in the carton are consistent;
measuring, via a scale, a weight of the egg in the carton to determine whether the weight of the egg is within an egg weight tolerance, the egg being lifted by an air vacuum suction cup; and
measuring, via a density sensor, a density of the egg to determine whether the egg is within a density tolerance.

20. The non-transitory computer-readable storage medium of claim 19, wherein performing the image analysis of the egg further comprises detecting a size and a shape of the egg in the carton and determining whether the egg meets a size and shape tolerance.
